(19) United States
(12) Patent Application Publication
(10) Pub. No.: US 2005/0267440 A1
(43) Pub. Date: Dec. 1, 2005

(54) Devices and methods for measuring and enhancing drug or analyte transport to/from medical implant

(76) Inventors: Stephen J. Herman, Andover, MA (US); Timothy Kreiger, Houston, TX (US); John T. Santini JR., North Chelmsford, MA (US); James H. Prescott, Cambridge, MA (US); Mark A. Staples, Cambridge, MA (US); Sara A. Lipka, Somerville, MA (US)

Correspondence Address: SUTHERLAND ASBILL & BRENNAN LLP, 999 PEACHTREE STREET, N.E., ATLANTA, GA, 30309, US

(21) Appl. No.: 11/142,629

(22) Filed: Jun. 1, 2005

Related U.S. Application Data
(60) Provisional application No. 60/575,946, filed on Jun. 1, 2004.
(60) Provisional application No. 60/635,780, filed on Dec. 13, 2004.
(60) Provisional application No. 60/593,832, filed on Feb. 17, 2005.
(60) Provisional application No. 60/655,785, filed on Feb. 24, 2005.

(51) Int. Cl. A61M 31/00

(52) U.S. Cl. 604/501; 604/20; 604/891.1

(57) ABSTRACT

Methods and devices are provided for enhancing mass transport through any fibrous tissue capsule that may form around an implanted medical device following implantation. Methods and devices are also provided to enhance vascularization around the implanted device, which also will aid in mass transport to/from the device. The device preferably comprises multiple reservoirs containing (i) a drug formulation for short- or long-term, controlled drug delivery, (ii) sensors for sensing an analyte in the patient, or (iii) a combination thereof.

### CROSS-REFERENCE TO RELATED APPLICATIONS

This application claims the benefit of U.S. Provisional Applications No. 60/575,946, filed Jun. 1, 2004; No. 60/635,780, filed Dec. 13, 2004; No. 60/593,832, filed Feb. 17, 2005; and No. 60/655,785, filed Feb. 24, 2005. The applications are incorporated herein by reference in their entirety.

### BACKGROUND OF THE INVENTION

This invention is generally in the field of implantable medical devices. In particular, the invention relates to apparatus and methods for measuring and modulating mass transport of drug or analyte through a tissue capsule structure to/from an implanted medical device, and for controlling tissue/implant interactions for improved function, integration, and useful life of the implant.

A variety of medical devices have been or are being developed for implantation into human and animal patients. Examples include drug delivery devices, biosensors, orthopedic prosthesis, and the like. Implantation of medical devices can induce inflammation and fibrosis when the body responds to the foreign object. Fibrosis results in the formation of a fibrous tissue capsule in the proximity of the device. Such capsules can vary in their composition, including extent of vascularity, water and cellular content, and the degree of crosslinking of collagen, which is typically their primary material. Thickness of capsules can range from a few microns to several millimeters.

During the lifetime of an implanted drug delivery device or biosensor, the structure of the fibrous tissue capsule may change, and such changes may adversely affect the transport of drug from the device, or the transport of an analyte to the device. For instance, a drug that needs to be delivered as a daily bolus (e.g., pulse) to be effective, such as parathyroid hormone to treat osteoporosis, could have its release slowed to a sub-therapeutic, or even a detrimental, rate of release. In addition, drugs that are cleared rapidly from the circulation, such as prostacyclins, may not be able to achieve therapeutic concentrations if they are released through the tissue capsule too slowly. Similarly, a tissue capsule may slow the diffusion of analytes or other substances to sensors contained in or on the implanted device. Slowing the diffusion rate of an analyte to a sensor will increase the time required to detect changes in the analyte or decrease the sensitivity of the sensor, either of which may render the sensor ineffective for analyte or therapeutic drug monitoring. For example, a tissue capsule may slow the rate of glucose transport to a glucose sensor, which introduces a time lag and results in a discrepancy between the actual and measured glucose level in the body. If the time lag becomes too large, the measured glucose level is no longer indicative of the actual glucose level. In this case, if a Type I diabetic were to make decisions on insulin dosing using the measured glucose level, they would be at risk of over or under dosing themselves, which could lead to a dangerous condition such as hypoglycemia.

It therefore would be desirable to provide methods, devices, compositions, or combinations thereof, to negate the diffusion rate-slowing effect of tissue capsules, for example so that effective drug release rates can be maintained over time from an implanted drug delivery device or so that implanted sensors can maintain their effectiveness.

Researchers have attempted to modify the structure of tissue capsules using various means as a way of characterizing and improving molecular transport through capsules. Current methodologies have been more or less limited to (1) in vitro tests (e.g., where the tissue capsule is removed from the animal, placed in a diffusion cell, and the transport through the ‘non-living’ capsule is measured) or (2) infusion of markers into the animal (e.g., the marker is infused into the animal, the animal is sacrificed, the tissue capsule is removed and frozen, and the capsule is analyzed for marker content and location) limiting analysis to only one time point per animal, which is highly inefficient and wasteful. These methods do not allow multiple or real time quantitative measurements to occur in situ or in vivo, which would provide the most realistic and reliable data. There remains a need to improve sensor biocompatibility and long term reliability and functionality, and to this end there remains a need to obtain in situ measurements of molecular transport across tissue capsules.

### SUMMARY OF THE INVENTION

Methods and devices have been developed for enhancing mass transport through fibrous tissue capsules that may form around an implanted medical device following implantation, for enhancing vascularization around the implanted device which also will aid in mass transport to/from the device, or both.

In one embodiment, a method is provided for enhancing the transport of a drug from an implanted drug delivery device across a tissue capsule. In this embodiment, the method includes controllably releasing a drug formulation from a plurality of discrete reservoirs located in medical device implanted in a patient; and controllably releasing an effective amount of a transport enhancer from said medical device implanted in a patient, to facilitate transport of the released drug formulation through a fibrous tissue capsule, if any, which exists around the device at the site of implantation.

In various embodiments, the release of the enhancing agent may be from one or more reservoirs located in the device, from a surface coating on the device, or from both of these locations. Release of the transport enhancer may occur concurrently with or temporally separate from release of the drug formulation. Release of the transport enhancer may occur continuously or at discrete intervals.

In one embodiment, the drug formulation further comprises the transport enhancer, and the drug formulation and the transport enhancer are released from the same reservoirs.

In one embodiment, the transport enhancer comprises a solvent or co-solvent for the drug. In another embodiment, the transport enhancer comprises a surfactant. Dimethylsulfoxide or N-methylpyrrolidone are examples. In still another embodiment, the drug molecules comprises charged molecules and the transport enhancer comprises ion-pairing counter-ions.

In one embodiment, the transport enhancer comprises molecules which dissolve or degrade components of the tissue capsule. Examples include collagenase, thrombin, fibrinolysin, hyaluronidase, trypsin, and combinations thereof.

In one embodiment, the device further includes means for mechanically driving the drug formulation out of the reservoir and through the tissue capsule. For example, the means for mechanically driving the drug formulation may include a piston, a water-swellable material, or a combination thereof.

In still another embodiment, the device further includes an angiogenic coating or angiogenic molecules for release. Vascular endothelial growth factor is an example of such a material. In another embodiment, the device further includes an anti-inflammatory agent, which is released from the reservoirs or from a coating on the device or both from the reservoirs and the coating. Dexamethasone is an example of such an agent.

In another aspect, a method is provided for enhancing the transport of drug from an implanted drug delivery device and across a tissue capsule, wherein the method includes the steps of controllably releasing a drug formulation, which comprises charged drug molecules, from a plurality of discrete reservoirs of a medical device implanted into a patient, the release of the drug and the release of the enhancing agent being from one or more reservoirs located in the device; and utilizing an electromotive method to enhance transport of the charged drug molecules through a tissue capsule, if any, surrounding the implanted medical device. In one example, the electromotive method includes iontophoresis. In one embodiment, an external surface of the medical device is charged by an electronic component therein, or thereon, creating a driving force effective to propel the drug molecules through tissue capsule surrounding the implanted medical device.

In another aspect, a method is provided for enhancing the transport of an analyte to a sensor device implanted in a patient. In one embodiment, the method includes the step of controllably releasing an effective amount of a transport enhancer from the implanted sensor device, wherein the device has a plurality of discrete reservoirs having sensors located therein. In one embodiment, the device further includes reservoir caps, and means for rupturing the reservoir caps.

In another aspect, an implantable medical device is provided that includes a body portion; two or more reservoirs located in and defined by the body portion; reservoir contents in the reservoirs; and means for enhancing mass transport, of all or a portion of the reservoir contents or of an environmental component intended for contact with all or a portion of the reservoir contents, through any fibrous tissue capsule that may form around the device following implantation. In one embodiment, the reservoir contents include a drug formulation. In another embodiment, the reservoir contents include a sensor or sensor component. In one embodiment, the means for enhancing mass transport includes a transport enhancer, an electromotive device, a positive displacement mechanism, or a combination thereof. The device optionally can include an angiogenic coating or angiogenic molecules for release. For example, the angiogenic coating, angiogenic molecules for release, or both, may include a vascular endothelial growth factor. In one embodiment, the device further includes an anti-inflammatory agent, which is released from the reservoirs or from a coating on the device or both from the reservoirs and the coating.

In another aspect, an implantable device is provided for testing drug or analyte transport through a tissue capsule. In one embodiment, the device includes a primary body having an outer surface, a perfusate fluid inlet, a perfusate fluid outlet, and a fluid conduit extending between the inlet and the outlet; a substrate attached to the primary body; at least one reservoir defined in and extending through the substrate, the reservoir having a first opening in the fluid conduit and a second opening which can be open to the outer surface of the device; at least one reservoir cap covering the second opening of the reservoir; and means for selectively disintegrating or removing the reservoir cap. The device typically would include a first flexible tubing connected to the perfusate fluid inlet, a second flexible tubing connected to the perfusate fluid outlet, and a means for flowing perfusate thorough the fluid conduit and the flexible tubings. In one embodiment, the device further includes a semipermeable barrier structure blocking bulk fluid flow through one or both of the reservoir openings following reservoir cap disintegration or removal.

### BRIEF DESCRIPTION OF THE FIGURES

FIG. 1 is a cross-sectional and perspective view of one embodiment of a device body with reservoirs and reservoir caps for opening by electrothermal ablation.

FIG. 2 is a cross-sectional view of a single reservoir of a device undergoing a process for loading the reservoir with a drug formulation and transport enhancing solvent.

FIG. 3 is a cross-sectional and partial view of one embodiment of implanted drug delivery device using electromotive driving means to drive a charged drug out of the device reservoirs and into/through a surrounding fibrous tissue capsule and microvasculature.

FIG. 4 is a perspective and partially exploded view of one embodiment of an implantable multi-reservoir medical device, as described herein.

FIG. 5 is a perspective view of a second embodiment of an implantable multi-reservoir medical device, as described herein.

FIG. 6 is a perspective and partially exploded view of one embodiment of an implantable multi-reservoir medical device having a protective mesh structure over the reservoir caps.

FIGS. 7A-B are cross-sectional and perspective views illustrating prior art drug and analyte perfusion processes through a semi-permeable membrane in tube form.

FIG. 8A is a perspective and cross-sectional view, and FIG. 8B is a cross-sectional view, of one embodiment of a testing device for use in measuring cross-tissue capsule transport.

FIG. 9 is a cross-sectional view of another embodiment of a testing device, which includes a semi-permeable tube inside the primary perfusate flow tube, for use in measuring cross-tissue capsule transport.

FIG. 10 is a cross-sectional view of another embodiment of a testing device, which includes a semi-permeable plug disposed in the reservoir opening, for use in measuring cross-tissue capsule transport.

FIG. 11 is a plan view of one embodiment of a testing device described herein for in vivo measurement of cross-tissue capsule transport

FIG. 12 is a perspective and cross-sectional view of still another embodiment of a testing device, which includes a plurality of individually openable reservoirs, for use in measuring cross-tissue capsule transport.

FIG. 13 is a perspective view of yet another embodiment of a testing device, which includes a plurality of individually openable reservoirs, for use in measuring cross-tissue capsule transport.

FIG. 14 is a cross-sectional view of the testing device shown in FIG. 13 being used to measure analyte flow through a tissue capsule.

FIG. 15 is a perspective view of a laboratory equipment set up/process for leak testing one of the testing devices for use in measuring cross-tissue capsule transport.

FIG. 16 is a perspective view of a laboratory equipment set up/process for in vitro testing one of the testing devices for use in measuring cross-tissue capsule transport.

### DETAILED DESCRIPTION OF THE INVENTION

In one aspect, methods and devices have been developed for enhancing mass transport through any fibrous tissue capsule that may form around an implanted medical device following implantation, and/or for enhancing vascularization around the implanted device, which also will aid in mass transport to/from the device.

In one embodiment, an implantable medical devices is provided that include a body portion; one or more reservoirs located in and defined by the body portion; reservoir contents; and a means for enhancing mass transport through any fibrous tissue capsule that may form around the device following implantation. Methods and devices are provided to enhance the transport of drug molecules and analytes across tissue capsules expected to develop around these devices following their implantation into a patient, and to reduce capsule tissue growth in the first instance.

In preferred embodiments, the device comprises a plurality of reservoirs, the contents of which may contain (i) a drug formulation for short- or long-term, controlled drug delivery, (ii) sensors for analyte or therapeutic drug monitoring, or (iii) both drug and sensors. In one embodiment, the device includes a drug formulation stored in and selectively released from the reservoirs and the means for enhancing mass transport of drug (released from the reservoirs) across and out of the tissue capsule. In another embodiment, the device includes a sensor and the means for enhancing mass transport enhances the transport of an analyte across the tissue capsule.

In another aspect, devices and methods have been developed for isolating the effect of tissue encapsulation. The devices advantageously allow access to the inside of intact tissue capsules in situ (in the animal), which, significantly, makes it possible to obtain detailed in situ measurements of molecular transport across tissue capsules. The devices will allow one to assess methods for modifying/modulating the properties or structure of the tissue capsule (e.g. thickness, vascularity, density, porosity, permeability, etc.) and to make quantitative comparisons of different strategies for improving transport—for example comparing two tissue capsules that have been formed in different ways or under the influence of different conditions. The devices also permit one to test different materials or device configurations. In one embodiment, the purpose of accessing the inside of the capsule is to test a device. For instance, one could compare devices that include means of opening a pathway into the device at a specific time, e.g., by mechanically rupturing, by electrochemically or electrothermally disintegrating, or by otherwise removing, a reservoir cap from an opening in the device body. In another embodiment, the purpose is to test a bulk material that is being considered for an implant material and observe what kind of capsule forms and whether such a material/device possibly would be useful as or in a drug delivery or biosensing device.

As used herein, the terms “comprise,” “comprising,” “include,” and “including” are intended to be open, non-limiting terms, unless the contrary is expressly indicated.

The Implantable Medical Device and Components Thereof

The medical device includes a body portion; one or more reservoirs located in and defined by the body portion; reservoir contents; and a means for enhancing mass transport (of all or a portion of the reservoir contents or of an environmental component intended for contact with all or a portion of the reservoir contents) through any fibrous tissue capsule that may form around the device following implantation. In one embodiment, the reservoir contents comprise a drug formulation, the reservoirs store the drug formulation, and control means control release of the drug formulation therefrom. In another embodiment, the reservoir contents comprise a sensor, the reservoirs store and protect the sensor, and control means control the time at which the sensor is exposed to the body (e.g., to a physiological fluid in vivo).

The control means can take a variety of forms. In one embodiment, each reservoir has an opening covered by a reservoir cap that can be selectively ruptured (e.g., disintegrated) to initiate release of the drug from the reservoir. For example, the reservoir cap can comprise a metal film that is disintegrated by electrothermal ablation as described in U.S. Patent Application Publication No. 2004/0121486 A1. Other reservoir opening and release control methods are described in U.S. Patent Application Publication Nos. 2002/0072784 A1, 2002/0099359 A1, 2002/0187260 A1, 2003/0010808 A1, 2004/0106914 A1, and 2005/0055014 A1; and U.S. Pat. Nos. 5,797,898; 6,123,861; 6,527,762; 6,551,838; 6,773,429; 6,808,522 all of which are incorporated by reference herein.

Device Body and Reservoirs

The device comprises a body portion, i.e., a substrate, that includes one or more reservoirs. A reservoir is a well, a recess, or a cavity, located in a solid structure and suitable for containing a quantity of another material and/or a small device. In a preferred embodiment, the device includes a plurality of the reservoirs located in discrete positions across at least one surface of the body portion.

In various embodiments, the body portion comprises silicon, a metal, a ceramic, a polymer, or a combination thereof. Examples of suitable substrate materials include metals, ceramics, semiconductors, glasses, and degradable and non-degradable polymers. Preferably each reservoir is formed of hermetic materials (e.g., metals, silicon, glasses, ceramics) and is hermetically sealed by a reservoir cap. Biocompatibility of the substrate material is preferred for in vivo device applications. For biocompatible and non-biocompatible materials, the substrate, or portions thereof, may be coated, encapsulated, or otherwise contained in a biocompatible material, such as poly(ethylene glycol), polytetrafluoroethylene-like materials, diamond-like carbon, inert ceramics, titanium, and the like, before use. In one embodiment, the substrate is hermetic, that is impermeable (at least during the time of use of the reservoir device) to the molecules to be delivered and to surrounding gases or fluids (e.g., water, blood, electrolytes or other solutions). In another embodiment, the substrate is made of a material that degrades or dissolves over a defined period of time into biocompatible components. Examples of such materials include biocompatible polymers, such as poly(lactic acid)s, poly(glycolic acid)s, and poly(lactic-co-glycolic acid)s, as well as degradable poly(anhydride-co-imides).

The substrate may consist of only one material, or may be a composite or multi-laminate material, that is, composed of several layers of the same or different substrate materials that are bonded together. In one embodiment, the substrate comprises layers of silicon and Pyrex bonded together. In another embodiment, the substrate comprises multiple silicon wafers bonded together. In yet another embodiment, the substrate comprises a low-temperature co-fired ceramic (LTCC). In one embodiment, the body portion is the support for a microchip device. In one example, this substrate is formed of silicon.

The body portion can have a variety of shapes, or shaped surfaces. It can, for example, have a release side (i.e., an area having reservoir caps) that is planar or curved. The substrate may, for example, be in a shape selected from circular or ovoid disks, cylinders, or spheres. In one embodiment, the release side can be shaped to conform to a curved tissue surface or into a body lumen. In another embodiment, the back side (distal the release side) is shaped to conform to an attachment surface. In various embodiments, the body portion is in the form of a chip, a disk, a tube, or a sphere. The body portion can be flexible or rigid.

Total substrate thickness and reservoir volume can be increased by bonding or attaching wafers or layers of substrate materials together. The device thickness may affect the volume of each reservoir and/or may affect the maximum number of reservoirs that can be incorporated onto a substrate. The size and number of substrates and reservoirs can be selected to accommodate the quantity and volume of reservoir contents needed for a particular application, manufacturing limitations, and/or total device size limitations to be suitable for implantation into a patient, preferably using minimally invasive procedures.

The substrate can have one, two, or preferably many, reservoirs. In various embodiments, tens, hundreds, or thousands of reservoirs are arrayed across the substrate. For instance, one embodiment of an implantable drug delivery device includes between 250 and 750 reservoirs, where each reservoir contains a single dose of a drug for release, which for example could be released daily over a period of several months to two years. More or less frequent dosing schedules and shorter or longer treatment durations are possible. In one sensing embodiment, the number of reservoirs in the device is determined by the operation life of the individual sensors. For example, a one-year implantable glucose monitoring device having individual sensors that remain functional for 30 days after exposure to the body would contain at least 12 reservoirs (assuming one sensor per reservoir).

In one sensor embodiment, the distance between the sensor surface and the reservoir opening means is minimized, preferably only a few microns. In this case, the volume of the reservoir is primarily determined by the surface area of the sensor. For example, the electrodes of a typical enzymatic glucose sensor may occupy a space that is 400 μm by 800 μm.

In one embodiment, the reservoirs are microreservoirs. As used herein, the term “microreservoir” refers to a concave-shaped solid structure suitable for releasably containing a material, wherein the structure is of a size and shape suitable for filling with a microquantity of the material, which comprises a drug. In one embodiment, the microreservoir has a volume equal to or less than 500 μL (e.g., less than 250 μL, less than 100 μL, less than 50 μL, less than 25 μL, less than 10 μL, etc.) and greater than about 1 nL (e.g., greater than 5 nL, greater than 10 nL, greater than about 25 nL, greater than about 50 nL, greater than about 1 μL, etc.). The shape and dimensions of the microreservoir can be selected to maximize or minimize contact area between the drug material and the surrounding surface of the microreservoir. As used herein, the term “microquantity” refers to small volumes between 1 nL and 10 μL. In one embodiment, the microquantity is between 1 nL and 1 μL. In another embodiment, the microquantity is between 10 nL and 500 nL.

In other embodiments, the reservoirs are larger than microreservoirs and can contain a quantity of drug formulation larger than a microquantity. For example, the volume of each reservoir can be greater than 10 μL (e.g., at least 20 μL, at least 50 μL, at least 100 μL, at least 250 μL, etc.) and less than 10,000 μL (e.g., less than 5000 μL, less than 1000 μL, less than 750 μL, less than 500 μL, less than 100 μL, etc.). These may be referred to as macro-reservoirs and macro-quantities, respectively. Unless explicitly indicated to be limited to either micro- or macro-scale volumes/quantities, the term “reservoir” is intended to include both.

Reservoirs can be fabricated in a structural body portion using any suitable fabrication technique known in the art. Representative fabrication techniques include MEMS fabrication processes or other micromachining processes, various drilling techniques (e.g., laser, mechanical, and ultrasonic drilling), and build-up techniques, such as LTCC (low temperature co-fired ceramics), as well as molding processes. See, for example, U.S. Pat. Nos. 6,123,861 and 6,808,522, as well as U.S. Patent Application Publication Nos. 2004/0106914 and 2005/0055014. The surface of the reservoir optionally can be treated or coated to alter one or more properties of the surface. Examples of such properties include hydrophilicity/hydrophobicity, wetting properties (surface energies, contact angles, etc.), surface roughness, electrical charge, release characteristics, and the like.

In one embodiment, the device comprises a microchip chemical delivery device. In another embodiment, the device includes polymeric chips or devices composed of non-silicon based materials that might not be referred to as “microchips.” Examples of various substrate and device configurations are described in U.S. Patent Application Publication No. 2004/0121486 A1. In one embodiment, the device comprises an osmotic pump, for example, the DUROS™ osmotic pump technology (Alza Corporation) included in commercial devices such as VIADUR™ (Bayer Healthcare Pharmaceuticals and Alza Corporation). In another embodiment, the device comprises a LTCC body. In one embodiment, the body portion is the support for a microchip device. In one example, this substrate can be formed of silicon.

Means For Enhancing Mass Transport

The implantable device may include one or a combination of components useful for enhancing the rate of mass transport through a tissue capsule. These include the use of enzymes, co-solvents, surfactants, or combinations thereof, useful in making highly concentrated, stable formulations, counter-ion drug formulations, enzymatic degradation, and electromotive devices. Another means for enhancing mass transport from the device reservoirs and through a tissue capsule includes the positive displacement and/or accelerated release techniques described in PCT WO 2004/026281 A1. Yet another means for enhancing mass transport involves enhancing the vascularity of the tissue capsule, such as with one or more angiogenic agents coated on or released from the device, which will facilitate drug or analyte transport therethrough. In various embodiments, combinations of these different means, materials, and techniques are used to enhance transport of drug or analyte through the tissue capsule.

In one embodiment, the device releases from the reservoirs, and/or is coated with, one or more anti-inflammatory agents. In one specific embodiment, the anti-inflammatory agent is dexamethasone. In these embodiments, the anti-inflammatory agent reduces inflammation following implantation, which can decrease the overall thickness of the fibrous capsule. In another specific embodiment, the device releases from the reservoirs, and/or is coated with, a combination of dexamethasone and VEGF, which can reduce inflammation and increase vascularity around the implanted device. See Norton, et al., “Dual Release of VEGF and Dexamethasone from Microspheres Incorporated in Anti-fouling Hydrogels” p. 357, Proceedings 7^{th} World Biomaterials Congress (Sydney, Australia) May 2004.

As used herein, the term “transport enhancers” refers to and includes solvents, co-solvents, and surfactants that alter tissue permeability, and enzymes that degrade tissue capsules, and thereby help drug molecules to penetrate the tissue capsule and reach their targets at effective (e.g., therapeutic) concentrations and rates or help analytes penetrate the tissue capsule to reach a sensor material at clinically/diagnostically useful concentrations and rates.

Solvent/Surfactant Formulations

In one embodiment, the means for enhancing mass transport comprises reservoir contents that include a material effective to alter tissue capsule permeability. Altered permeability of the tissue capsule can permit greater mass transport of drug or analyte therethrough. In one embodiment, the drug formulation includes one or more solvents, co-solvents, surfactants, or combinations thereof, useful in making highly concentrated, stable formulations and/or useful in altering tissue permeability. The small dose volumes of the reservoir devices advantageously permit an active ingredient, i.e., a drug, to be dissolved or physically mixed with powerful solvents, co-solvents, and/or surfactants that otherwise would cause tissue irritation or damage if used in larger volumes. Examples of such solvents and the Permitted Daily Exposure limits are provided in ICH Guideline Q3C. Although these limits were intended for residual solvents remaining in drug product from processing operations, many of the listed solvents could be contained in device reservoirs without exceeding these recommended limits. For example, the Center for Drug Evaluation and Research (CDER) at the United States Food and Drug Administration (FDA) has classified nitromethane as a Class 2 solvent with a Per Day Exposure (PDE) limit of 500 μg (440 nL), which is the most stringent, recommended limit for Class 2 solvents. In one embodiment of the present devices, each reservoir contains 200 nL of a Class 2 Solvent, and if only one reservoir is released per day, then exposure to the solvent would be less than half the PDE.

Representative examples of suitable solvents include dimethylsulfoxide (DMSO), N-methylpyrrolidone (NMP), as well as Dimethylpyrrolidone (DMP), dimethylformamide (DMF), dimethylacetamide, acetonitrile, and other polar, aprotic solvents that alter the structure of collagen and collagen networks. In one embodiment, the small volumes per dose (≦200 nL/dose) are well below the daily exposure limits for the Class 2 solvents. Representative examples of suitable surfactants include polysorbates, Spans, monoalkylpolyoxythenes, dialkylpolyoxyethylenes, polyoxyethylene monoesters, polyoxyethylene diesters, and polyoxyethylene-polyoxyethylene block copolymers. To derive a suitable formulation one can, for example, (1) identify a solvent or surfactant, alone or in combination, that provides the required drug solubility and stability, and (2) screen for penetration enhancement by comparing the rate of drug movement across a sample of the appropriate capsular tissue using a device such as a Franz cell or multi-well microdialysis unit (as described below). By selecting appropriate solvents, co-solvents, and surfactants, one is able to produce small molecule, peptide, and protein drug formulations that are highly concentrated (for example, ≧100 mg/mL or 10% w/v or 10% v/v) and stable at body temperature.

Furthermore, the use of concentrated solutions of drug enables one to limit reservoir size and thus device size, which in turn advantageously could limit implant mobility, which may reduce tissue capsule growth. High concentrations advantageously can keep the implantable device relatively small in size (because there is no need for the device to be sized to contain extra solvent volume), which can make their placement in the patient safer and less obtrusive. In addition, a smaller implant with suture anchoring desirably would be less mobile than a larger device. Mobility has been identified as a contributor to tissue capsule growth.

In one embodiment, a transport enhancer is released from some of the reservoirs. The reservoirs can contain one or more transport enhancers alone or in combination with a drug formulation for release. In another embodiment, the device includes a coating, such as a controlled-release coating, comprising the transport enhancer. In one variation, the transport enhancer is provided in the device in one or more reservoirs separate from the reservoirs containing the drug formulation for release. For example, in a device comprising a substrate having an array of reservoirs located therein, the transport enhancer and the drug formulation could be stored in alternating reservoirs, and release of the transport enhancer could be directed to closely or immediately precede release of the drug formulation. Alternatively, release of the transport enhancer could be simultaneous with, or could follow, release of the drug formulation.

In one embodiment, a transport enhancer is contained in one or more reservoirs near to, but separate, from reservoirs containing glucose sensors. The release of the transport enhancer can be timed with respect to exposure of the glucose sensor, or the transport enhancer could be released from the reservoirs on a regular schedule. The latter situation would work to keep the capsule at the same level of permeability over a long implantation period.

Enzymatic Degradation

In yet another approach, the implant device include molecules that are known to dissolve or degrade the components of a tissue capsule can be used to decrease the transport limiting effects of the tissue capsule. For example, a composition comprising collagenase, thrombin, fibrinolysin, trypsin, hyaluronidase, or a combination thereof, could be included in, on, or with the device. The enzyme could be packaged in reservoirs, attached to the surface of the device, or incorporated into a release-modifying matrix.

In one embodiment, the enzyme for enhancing transport is contained in one or more reservoirs of the device. Reservoirs can be opened prior to releasing a drug from one or more neighboring reservoirs, such that the enzyme dissolves at least a portion of the capsule near the drug reservoir, thereby reducing the capsule's barrier properties and minimizing the capsule's effect on the drug release rate when the drug reservoir is opened. A similar strategy can be used with biosensing devices to increase the permeability of a capsule to an analyte such as glucose. If the tissue capsule comprises significant amounts of fibrin or fibrinogen, then the device could release thrombin, fibrinolysin, trypsin, or another effective enzyme.

Counter-Ion Drug Formulations

In another embodiment, the drug is comprised of charged molecules, and ion-pairing counterions are included in the drug formulation to modulate drug transport through a tissue capsule. The ability of counterions to alter binding of charged molecules, including peptides and drugs, to materials is well known as illustrated by RP-HPLC and the influence of the Hofmeister series ions on protein structure and ion exchange chromatography retentions. Ion pair formation has been used in organic chemistry to facilitate the movement of reactants and products between organic and aqueous phases to provide in situ reaction compartmentalization. Ion pairing of drugs to vary their performance by changing lipid solubility, particle size, or micelle formation has been examined (Choi, et al., Int'l J Pharmaceutics 203(1):193-202 (2000); Kendrick, et al., Arch. Biochem. Biophys. 347:113-18 (1997); Meyer, et al., Pharm. Res. 15(2):188-93 (1998)). It may also be possible to disrupt or modify the hydrogen bonds or ionic bonds within the capsule's collagen structure by interaction or exchange of the drug counterion with the collagen.

Electromotive Devices and Methods

In yet another embodiment, the drug can be charged and iontophoretic or other electromotive methods known in the art are used to enhance transport through a tissue capsule. For example, an exposed surface of the implanted device can be charged by its internal electronics to create a driving force that would propel drug molecules possessing the same charge through a capsule. In one embodiment, a pair of oppositely charged electrodes are located on the same device or surface of the device, but are positioned sufficiently far apart from one another so that the path of least electrical resistance through the tissue barrier. In another embodiment, a counter electrode, separate from the drug delivery component, is located outside of the tissue capsule.

Positive Displacement Devices and Methods

In one embodiment, positive displacement mechanisms are used to drive a drug formulation out of the reservoirs. These same mechanisms can also be employed to drive or push the drug formulation through the tissue capsule. In one embodiment, an osmotic pressure generating material or other swellable material drives a piston to force a drug formulation out of the reservoir. This and other embodiments are detailed in PCT WO 2004/026281 A1.

In one embodiment, the reservoir contents are sealed in a gas-tight or hermetic reservoir under compression or sealed under conditions to create a positive pressure internal environment so that contents are expelled upon reservoir cap activation.

In another embodiment, the device comprises three substrate portions packaged together, with the reservoir spaces aligned and defining three compartments (reservoirs). The bottom compartment includes a dry swellable gel, the middle compartment includes a liquid material (or at least liquid at body temperature), and the top compartment includes a drug formulation for release. Membranes (or reservoir caps) between the layers separate the adjacent compartments, until release is intended. After or simultaneously with disintegration of a reservoir cap over the exterior opening of a drug-loaded reservoir, the membrane over the swellable gel is opened to allow liquid from the middle compartment to contact the gel, causing the swellable gel to expand. The gel material(s) would be selected to have an expanded volume that exceeded the combined volumes of the reservoir compartments. Optionally, expansion could be augmented by a controlled temperature change (e.g., with a resistive heater element disposed in the reservoir) where the gel is one known in the art to expand/contract with temperature. The membrane over the liquid is opened to allow the swelling gel to displace the drug formulation from the top compartment.

Angiogenic Materials and Agents

In one embodiment, the device is provided with a coating that comprises one or more angiogenic materials or factors to promote vascularization around the implanted device. This would be useful with both implantable drug delivery devices and implantable analyte monitoring devices (e.g., glucose sensors). As used herein, the term “angiogenic” refers to a material or molecules that promote and maintain the development of blood vessels and microcirculation around the implanted device. In one embodiment example, the device releases or is coated with a vasoinductive or angiogenic agent such as a vascular growth factor. Suitable growth factors of this type include as vascular endothelial growth factor (VEGF), platelet growth factor, vascular permeability factor, fibroblast growth factor, and transforming growth factor beta.

In another embodiment, the device includes an exterior membrane or coating layer that itself exhibits angiogenic properties. These layers can be made for example of expanded polytetrafluoroethylene (ePTFE), hydrophilic polyvinylidene fluoride, mixed cellulose esters, and/or other polymers.

Device Surface Modification and Scaffolding

In still another embodiment, the implantable medical device includes tissue scaffolding or other physical surface modification effective to promote transport over a baseline case of an unmodified surface. Underlying material properties that may affect tissue capsule deposition include alternations in surface hydrophilicity, surface area, porosity (both percent and diameter of individual pores), and degree of nanometer-scale roughness. These concepts are understood in the art, and those in the art can adapt conventional approaches for use with the implantable multi-reservoir devices described herein. As used herein, the term “scaffolding” refers to a three-dimensional surface topography that may include nanometer scale features such as roughness or porosity. That is, the topography may serve as a scaffold on which cell adhesion occur in a controlled fashion, reducing formation of avascular tissue and resulting n a tissue quality more likely to permit cross-tissue transport. The surface could, in turn, be modified by deposition of an additional layer of a different material or with chemical or biochemical decoration that could affect cell adhesion.

Reservoir Control Means

The reservoir control means comprises the structural component(s) for controlling the time at which release or exposure of the reservoir contents is initiated. In a preferred embodiment, the reservoir control means includes reservoir caps and the hardware, electrical components, and software needed to control and deliver electric energy from a power source to selected reservoir(s) for actuation, e.g., reservoir opening.

Reservoir Caps

As used herein, the term “reservoir cap” includes a discrete membrane or other structure suitable for separating the contents of a reservoir from the environment outside of the reservoir. It generally is self-supporting across the reservoir opening, although caps having additional structures to provide mechanical support to the cap can be fabricated. See, e.g., U.S. Pat. No. 6,875,208. Selectively removing the reservoir cap or making it permeable will then “expose” the contents of the reservoir to the environment (or selected components thereof) surrounding the reservoir. In preferred embodiments, the reservoir cap is selectively disintegrated. As used herein, the term “disintegrate” includes degrading, dissolving, rupturing, fracturing or some other form of mechanical failure, as well as a loss of structural integrity due to a chemical reaction (e.g., electrochemical degradation) or phase change (e.g., melting) in response to a change in temperature, unless a specific one of these mechanisms is indicated. In several preferred embodiments, removal of the reservoir cap primarily involves a chemical reaction or phase change component, as opposed to a mechanically activated rupturing (e.g., relying on prestressed, brittle membranes being fractured by a mechanical force from a piezoelectric member, or gas pressure generated mechanical rupture).

In one specific embodiment, the disintegration is by an electrochemical activation technique, such as described in U.S. Pat. No. 5,797,898, which is incorporated herein by reference. For example, the reservoir cap can be a thin metal film which is impermeable to the surrounding environment (e.g., body fluids or another chloride containing solution). In one variation, a particular electric potential is applied to the metal reservoir cap, which is then oxidized and disintegrated by an electrochemical reaction, to release the drug from the reservoir. Examples of suitable reservoir cap materials include gold, silver, copper, and zinc.

In another specific embodiment, the disintegration is by thermal activation technique, such as described in U.S. Pat. No. 6,527,762, which is incorporated herein by reference. For example, the reservoir cap can be heated (e.g., using resistive heating from a separate resistive heater) to cause the reservoir cap to melt and be displaced from the reservoir opening, to open the reservoir. This latter variation could be used, for example, with reservoir caps formed of a metal or a non-metal material, e.g., a polymer. In yet another variation, the reservoir cap is formed of a polymer or other material that undergoes a temperature-dependent change in permeability such that upon heating to a pre-selected temperature, the reservoir is rendered permeable to the drug and bodily fluids to permit the drug to be released from the reservoir through the reservoir cap.

In a preferred embodiment, the “disintegration” is by an electro-thermal ablation technique, as described in U.S. Patent Application Publication No. 2004/0121486 A1, which is incorporated herein by reference. For example, the reservoir cap is formed of a conductive material, such as a metal film, through which an electrical current can be passed to electrothermally ablate it. Representative examples of suitable reservoir cap materials include gold, copper, aluminum, silver, platinum, titanium, palladium, various alloys (e.g., Au—Si, Au—Ge, Pt—Ir, Ni—Ti, Pt—Si, SS 304, SS 316), and silicon doped with an impurity to increase electrical conductivity, as known in the art. In one embodiment, the reservoir cap is in the form of a thin metal film. In one embodiment, the reservoir cap is part of a multiple layer structure, for example, the reservoir cap can be made of multiple metal layers, such as a multi-layer/laminate structure of platinum/titanium/platinum. The reservoir cap is operably (i.e., electrically) connected to an electrical input lead and to an electrical output lead, to facilitate flow of an electrical current through the reservoir cap. When an effective amount of an electrical current is applied through the leads and reservoir cap, the temperature of the reservoir cap is locally increased due to resistive heating, and the heat generated within the reservoir cap increases the temperature sufficiently to cause the reservoir cap to be electrothermally ablated and ruptured.

In a preferred embodiment, a discrete reservoir cap completely covers a single reservoir opening. In another embodiment, a discrete reservoir cap covers two or more, but less than all, of the reservoir's openings.

In passive release devices, the reservoir cap is formed from a material or mixture of materials that degrade, dissolve, or disintegrate over time, or that do not degrade, dissolve, or disintegrate, but are permeable or become permeable to drug or analyte molecules. Representative examples of reservoir cap materials include polymeric materials, and non-polymeric materials such as porous forms of metals, semiconductors, and ceramics. Passive semiconductor reservoir cap materials include nanoporous or microporous silicon membranes.

Characteristics can be different for each reservoir cap to provide different times of release of drug formulation. For example, any combination of polymer, degree of crosslinking, or polymer thickness can be modified to obtain a specific release time or rate. Any combination of passive and/or active release reservoir cap can be present in a single delivery device. For example, the reservoir cap can be removed by electrothermal ablation to expose a passive release system that only begins its passive release after the reservoir cap has been actively removed. Alternatively, a given device can include both passive and active release reservoirs.

In one embodiment, the device includes (i) active release reservoirs containing a drug formulation, and (ii) passive release reservoirs containing one or more transport enhancers. In one method with this embodiment, the transport enhancement molecules are continuously released from the passive release reservoirs to maintain a constant capsule permeability, while the active release reservoirs are opened on a schedule determined by the type of drug therapy prescribed by the physician.

In another embodiment, the device includes (i) active release reservoirs containing sensors, and (ii) passive release reservoirs containing one or more transport enhancers. In one method with this embodiment, the transport enhancement molecules are continuously released from the passive release reservoirs to maintain a constant capsule permeability, while the active release reservoirs are opened as needed (depending, for example, upon fouling of the sensor) or as dictated by a predetermined schedule.

In yet another embodiment, the device includes (i) active release reservoirs containing a drug formulation, and (ii) active release reservoirs containing one or more transport enhancers. In one method with this embodiment, the transport enhancement molecules are released periodically or on a schedule to coincide with or precede the opening of the drug-containing active release reservoirs, which are opened on a schedule, determined by the prescribed drug therapy.

Other Components

The control means can provide intermittent or effectively continuous release of the drug formulation and/or the transport enhancer, and/or selective exposure of sensors. The particular features of the control means depend on the mechanism of reservoir cap activation described herein. For example, the control means can include an input source, a microprocessor, a timer, a demultiplexer (or multiplexer), and a power source. As used herein, the term “demultiplexer” also refers to multiplexers. The power source provides energy to activate the selected reservoir, e.g., to trigger release of the drug formulation from the particular reservoir desired for a given dose. For example, the operation of the reservoir opening system can be controlled by an on-board microprocessor (e.g., the microprocessor is within an implantable or insertable device). The microprocessor can be programmed to initiate the disintegration or permeabilization of the reservoir cap at a pre-selected time or in response to one or more of signals or measured parameters, including receipt of a signal from another device (for example by remote control or wireless methods) or detection of a particular condition using a sensor such as a biosensor. In another embodiment, a simple state machine is used, as it typically is simpler, smaller, and/or uses less power than a microprocessor. The device can also be activated or powered using wireless means, for example, as described in U.S. 2002/0072784 A1 to Sheppard et al.

In one embodiment, the device includes a substrate having a two-dimensional array of discretely spaced reservoirs arranged therein, a drug formulation contained in the reservoirs, anode reservoir caps covering a semi-permeable membrane for each of the reservoirs, cathodes positioned on the substrate near the anodes, and means for actively controlling disintegration of the reservoir caps. The means includes a power source and circuitry to control and deliver an electrical potential; the energy drives a reaction between selected anodes and cathodes. Upon application of a potential between the electrodes, electrons pass from the anode to the cathode through the external circuit causing the anode material (reservoir cap) to oxidize and dissolve into the surrounding fluids, exposing and releasing the drug formulation. The microprocessor directs power to specific electrode pairs through a demultiplexer as directed by an EPROM, remote control, or biosensor.

In another embodiment, the activation energy initiates a thermally driven rupturing or permeabilization process, for example, as described in U.S. Pat. No. 6,527,762. For example, the means for controlling release can actively disintegrate or permeabilize a reservoir cap using a resistive heater. The resistive heater can cause the reservoir cap to undergo a phase change or fracture, for example, as a result of thermal expansion of the reservoir cap or release system, thereby rupturing the reservoir cap and releasing the drug from the selected reservoir. The application of electric current to the resistor can be delivered and controlled using components as described above for use in the electrochemical disintegration embodiment. For example, a microprocessor can direct current to select reservoirs at desired intervals.

In a preferred embodiment, control means controls electro-thermal ablation of the reservoir cap. For example, the drug delivery device could include a reservoir cap formed of an electrically conductive material; an electrical input lead connected to the reservoir cap; an electrical output lead connected to the reservoir cap; and a control means to deliver an effective amount of electrical current through the reservoir cap, via the input lead and output lead, to locally heat and rupture the reservoir cap, for example to release the drug formulation or expose the sensor located therein. In one embodiment, the reservoir cap and conductive leads are formed of the same material, where the temperature of the reservoir cap increases locally under applied current because the reservoir cap is suspended in a medium that is less thermally conductive than the substrate. Alternatively, the reservoir cap and conductive leads are formed of the same material, and the reservoir cap has a smaller cross-sectional area in the direction of electric current flow, where the increase in current density through the reservoir cap causes an increase in localized heating. The reservoir cap alternatively can be formed of a material that is different from the material forming the leads, wherein the material forming the reservoir cap has a different electrical resistivity, thermal diffusivity, thermal conductivity, and/or a lower melting temperature than the material forming the leads. Various combinations of these embodiments can be employed as described in U.S. Patent Application Publication No. 2004/0121486 A1.

The implantable devices typically are hermetically sealed, e.g., in a titanium encasement, which exposes substantially only the reservoir caps.

In one embodiment, the control means includes a microprocessor, a timer, a demultiplexer, and an input source (for example, a memory source, a signal receiver, or a biosensor), and a power source. The timer and demultiplexer circuitry can be designed and incorporated directly onto the surface of the microchip during electrode fabrication, or may be incorporated in a separate microchip. The microprocessor translates the output from memory sources, signal receivers, or biosensors into an address for the direction of power through the demultiplexer to a specific reservoir on the device. Selection of a source of input to the microprocessor such as memory sources, signal receivers, or biosensors depends on the microchip device's particular application and whether device operation is preprogrammed, controlled by remote means, or controlled by feedback from its environment (i.e., biofeedback). For example, a microprocessor can be used in conjunction with a source of memory such as erasable programmable read only memory (EPROM), a timer, a demultiplexer, and a power source such as a battery or a biofuel cell. A programmed sequence of events including the time a reservoir is to be opened and the location or address of the reservoir is stored into the EPROM by the user. When the time for exposure or release has been reached as indicated by the timer, the microprocessor sends a signal corresponding to the address (location) of a particular reservoir to the demultiplexer. The demultiplexer routes an input, such as an electric potential or current, to the reservoir addressed by the microprocessor.

Reservoir Contents

The reservoirs contain a drug formulation, a sensing device, a transport enhancer, or a combination thereof.

Drug

The drug formulation is a composition that comprises a drug. As used herein, the term “drug” includes any therapeutic or prophylactic agent (e.g., an active pharmaceutical ingredient or API). In one embodiment, the drug is provided in a solid form, particularly for purposes of maintaining or extending the stability of the drug over a commercially and medically useful time, e.g., during storage in a drug delivery device until the drug needs to be administered. The solid drug matrix may be in pure form or in the form of solid particles of another material in which the drug is contained, suspended, or dispersed. In one embodiment, the drug is formulated with an excipient material that is useful for accelerating release, e.g., a water-swellable material that can aid in pushing the drug out of the reservoir and through any tissue capsule over the reservoir.

The drug can comprise small molecules, large (i.e., macro-) molecules, or a combination thereof. In one embodiment, the large molecule drug is a protein or a peptide. In various other embodiments, the drug can be selected from amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants (e.g., LMWH, pentasaccharides), antibiotics (e.g., immunosuppressants), analgesic agents, and vitamins. In one embodiment, the drug is a protein. Examples of suitable types of proteins include, glycoproteins, enzymes (e.g., proteolytic enzymes), hormones or other analogs (e.g., LHRH, steroids, corticosteroids, growth factors), antibodies (e.g., anti-VEGF antibodies, tumor necrosis factor inhibitors), cytokines (e.g., α-, β-, or γ-interferons), interleukins (e.g., IL-2, IL-10), and diabetes/obesity-related therapeutics (e.g., insulin, exenatide, PYY, GLP-1 and its analogs). In one embodiment, the drug is a gonadotropin-releasing (LHRH) hormone analog, such as leuprolide. In another exemplary embodiment, the drug comprises parathyroid hormone, such as a human parathyroid hormone or its analogs, e.g., hPTH(1-84) or hPTH(1-34). In a further embodiment, the drug is selected from nucleosides, nucleotides, and analogs and conjugates thereof. In yet another embodiment, the drug comprises a peptide with natriuretic activity. In still another embodiment, the drug is selected from diuretics, vasodilators, inotropic agents, anti-arrhythmic agents, Ca⁺ channel blocking agents, anti-adrenergics/sympatholytics, and renin angiotensin system antagonists. In one embodiment, the drug is a VEGF inhibitor, VEGF antibody, VEGF antibody fragment, or another anti-angiogenic agent. Examples include an aptamer, such as MACUGEN™ (Pfizer/Eyetech) (pegaptanib sodium)) or LUCENTIS™ (Genetech/Novartis) (rhuFab VEGF, or ranibizumab), which could be used in the prevention of choroidal neovascularization. In yet a further embodiment, the drug is a prostaglandin, a prostacyclin, or another drug effective in the treatment of peripheral vascular disease.

In still another embodiment, the drug is an angiogenic agent, such as VEGF. In a further embodiment, the drug is an anti-inflammatory, such as dexamethasone. In one embodiment, a device includes both angiogenic agents and anti-inflammatory agents.

In various embodiments, the drug is a bone morphogenic protein (BMP), a growth factor (GF), or a growth or differentiation factor (GDF). Representative examples include BMP-2, OP-1 (osteogenic protein-1, i.e, BMP-7), morphogenic proteins (CDMP), osteogenin, BMP-2/4, BMP-3, BMP-9, BMP-10, BMP-15, and BMP-16, GDF-5 or rhGDF-5, Epidermal Growth Factors (EGF), Platelet-Derived Growth Factors (PDGF), Fibroblast Growth Factors (FGFs), Transforming Growth Factors α & β (TGF-α and TGF-β), Erythropoietin (EPO), Insulin-Like Growth Factor-I and -II (IGF-I and IGF-11), Tumor Necrosis Factors-α & -β (TNF-a and TNF-β), Colony Stimulating Factors (CSFs), and Neuronal Growth Factor (NGF).

The reservoirs in one device can include a single drug or a combination of two or more drugs, and/or two or more transport enhancers, and can further include one or more pharmaceutically acceptable carriers. Two or more transport enhancers, angiogenic agents, anti-inflammatory agents, or combinations thereof, can be stored together and released from the same one or more reservoirs or they can each be stored in and released from different reservoirs.

Excipients and Matrix Materials

The drug, the transport enhancer, or both, can be dispersed in a matrix material, to further control the rate of release of drug, transport enhancer, or both. This matrix material can be a “release system,” as described in U.S. Pat. No. 5,797,898, the degradation, dissolution, or diffusion properties of which can provide a method for controlling the release rate of the chemical molecules.

The release system may include one or more pharmaceutical excipients. Suitable pharmaceutically acceptable excipients include most carriers approved for parenteral administration. Other excipients may be used to maintain the drug in suspensions as an aid to reservoir filling, stability, or release. Depending on the properties of the drug, such excipients may be aqueous or non-aqueous, hydrophobic or hydrophilic, polar or non-polar, protic or aprotic. See, e.g., U.S. Pat. No. 6,264,990. The release system optionally includes stabilizers, antioxidants, antimicrobials, preservatives, buffering agents, surfactants, and other additives useful for storing and releasing molecules from the reservoirs in vivo.

The release system may provide a temporally modulated release profile (e.g., pulsatile release) when time variation in plasma levels is desired or a more continuous or consistent release profile when a constant plasma level as needed to enhance a therapeutic effect, for example. Pulsatile release can be achieved from an individual reservoir, from a plurality of reservoirs, or a combination thereof. For example, where each reservoir provides only a single pulse, multiple pulses (i.e. pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. Continuous release also can be controlled from reservoirs by incorporating a rate-limiting semi-permeable membrane in or over the reservoir opening(s). In addition, continuous release can be approximated by releasing several pulses of molecules in rapid succession (“digital” release). The active release systems described herein can be used alone or on combination with passive release systems, for example, as described in U.S. Pat. No. 5,797,898. For example, the reservoir cap can be removed by active means to expose a passive release system, or a given substrate can include both passive and active release reservoirs.

In one embodiment, the drug formulation within a reservoir comprises layers of drug and non-drug material. After the active release mechanism has exposed the reservoir contents, the multiple layers provide multiple pulses of drug release due to intervening layers of non-drug.

Sensing Device

In some embodiments, a sensing component or device may be provided in one, or preferably several, of the reservoirs of the device. In a preferred embodiment, two or more reservoirs contain a biosensor that can be used to detect the presence, absence, or change in a chemical or ionic species or energy at a site in vivo. For example, the sensor could monitor the concentration of glucose, urea, calcium, or a hormone present in the blood, plasma, interstitial fluid, or other bodily fluid of the patient.

Types of sensors include biosensors, chemical sensors, physical sensors, or optical sensors. Examples of biosensors that could be adapted for use in/with the reservoir devices described herein include those taught in U.S. Pat. No. 6,486,588; No. 6,475,170; and No. 6,237,398, which are incorporated herein by reference. Other sensing devices are described in U.S. Pat. No. 6,551,838 and in U.S. Patent Application Publication No. 2005/0096587 A1, which are incorporated herein by reference. As used herein, the term “biosensor” includes sensing devices that transduce the chemical potential of an analyte of interest into an electrical signal, as well as electrodes that measure electrical signals directly or indirectly (e.g., by converting a mechanical or thermal energy into an electrical signal). For example, the biosensor may measure intrinsic electrical signals (EKG, EEG, or other neural signals), pressure, temperature, pH, or loads on tissue structures at various in vivo locations. The electrical signal from the biosensor can then be measured, for example by a microprocessor/controller, which then can transmit the information to a remote controller, another local controller, or both. For example, the system can be used to relay or record information on the patient's vital signs or the implant environment, such as drug concentration.

Several options exist for receiving and analyzing data obtained with the sensing device. Devices may be controlled by local microprocessors or remote control. Biosensor information may provide input to the controller to determine the time and type of activation automatically, with human intervention, or a combination thereof. For example, the operation of an implantable drug delivery system (or other controlled release/controlled reservoir exposure system) can be controlled by an on-board microprocessor (i.e., within the package of the implantable device). The output signal from the device, after conditioning by suitable circuitry if needed, will be acquired by the microprocessor. After analysis and processing, the output signal can be stored in a writeable computer memory chip, and/or can be sent (e.g., wirelessly) to a remote location away from the implantable device. Power can be supplied to the implantable device locally by a battery or remotely by wireless transmission. See, e.g., U.S. Patent Application Publication No. 2002/0072784.

In one embodiment, a device is provided having reservoir contents that include drug molecules for release and a sensor/sensing component. For example, the sensor or sensing component can be located in a reservoir or can be attached to the device substrate. The sensor can operably communicate with the device, e.g., through a microprocessor, to control or modify the drug release variables, including dosage amount and frequency, time of release, effective rate of release, selection of drug or drug combination, and the like. The sensor or sensing component detects (or not) the species or property at the site of in vivo implantation and further may relay a signal to the microprocessor used for controlling release from the device. Such a signal could provide feedback on and/or finely control the release of a drug.

In one embodiment, the device contains one or more sensors for use in glucose monitoring and insulin control. Information from the sensor could be used to actively control insulin release from the same device or from a separate insulin delivery device (e.g., a conventional insulin pump, either an externally worn version or an implanted version).

Use of the Implantable Medical Device

The implantable medical device can take a variety of forms and be used in a variety of therapeutic and/or diagnostic applications. The implantable device comprising the reservoir means, reservoir contents, reservoir control means, and transport enhancing means can be integrated into another medical system or device. Examples include implantable controlled drug delivery devices, drug pumps (such as an implantable osmotic or mechanical pump), and combinations thereof.

Methods of using and operating the devices are further described in U.S. Pat. Nos. 5,797,898; 6,527,762; 6,491,666; 6,551,838; and 6,875,208; as well as U.S. Patent Application Publication Nos. 2002/0099359 A1, 2004/0082937 A1, 2004/0127942 A1, 2004/0106953 A1, and 2005/0096587 A1, all of which are incorporated by reference herein.

### Illustrative Embodiments

The present devices and methods can be further understood with reference to the appended drawings, where like numbers refer to the same device or component.

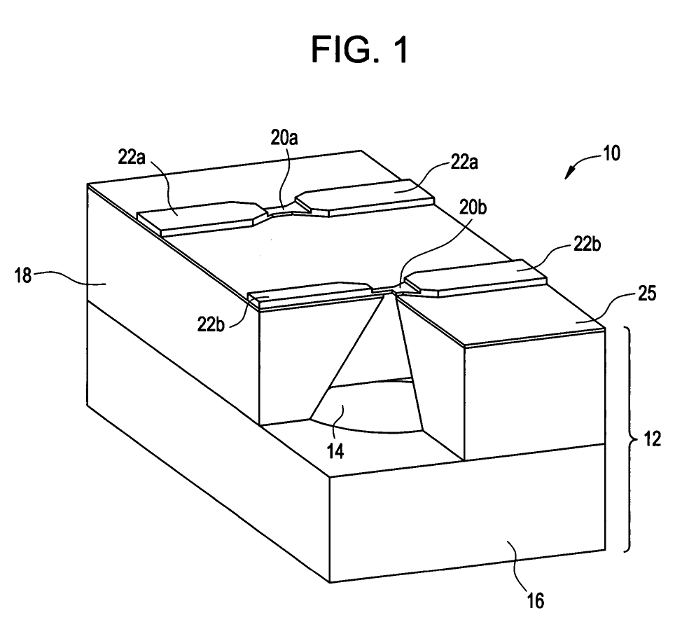

FIG. 1 shows one embodiment of the reservoirs and reservoir caps of a multi-reservoir, implantable medical device. Device 10 (shown only in part) comprises body portion 12, which includes a first substrate portion 18 and a second substrate portion 16. Reservoirs 14 are defined in the body portion. (Two are located in the body portion in this illustration, but only one can be seen from the cut-away of part of the first substrate portion.) The release opening of the reservoirs are covered by reservoir caps 20a and 20b. Metal conductors 22a and 22b are electrically connected to the reservoir caps, for delivering electric current to the reservoir caps (for reservoir opening by electrothermal ablation). Dielectric layer 25 is provided on the outer surface of the first substrate portion and is underneath the conductors. The reservoirs include reservoir contents (not shown), such as a drug formulation or sensor, and the device includes one or more means to enhance cross-tissue capsule transport.

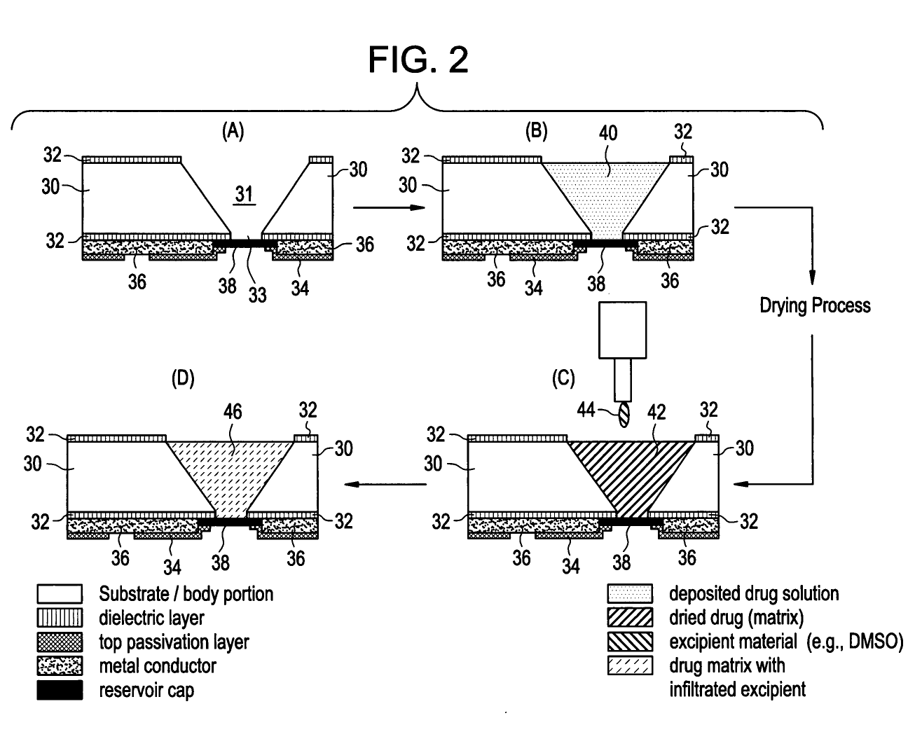

FIG. 2 shows in a cross-sectional view one embodiment of a reservoir in the body portion and shows the reservoir being loaded with a drug formulation. The substrate 30 includes reservoir 31, which has release opening 33 covered by reservoir cap 38. (Although not shown here, the wider fill-side of the reservoir will be sealed following completion of the drug loading and formulating processes described herein.) Metal conductors 36 can deliver electric current through reservoir cap 38 at the desired time of opening the reservoir to initiate release of drug formulation 46. Dielectric layer 32 and top passivation layer 34 are also shown. The drug formulation 46 is loaded into the reservoir by depositing a fluid drug solution or suspension 40 into the reservoir, lyophilizing or drying the solution to leave a dried drug matrix 42 in the reservoir, and then back-filling the matrix with an excipient material 44, such as a solvent that enhances tissue capsule transport.

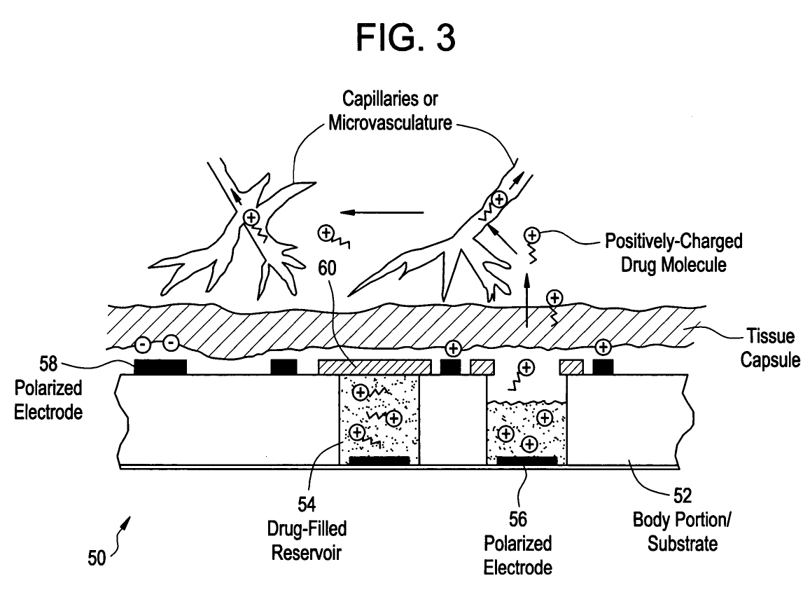

FIG. 3 illustrates one embodiment of an implanted drug delivery device 50, which includes body portion (or substrate) 52, reservoirs 54 loaded with a drug formulation and covered by reservoir caps 60. A polarized electrode 56 is located inside the reservoirs distal the opening. Electrode 56 is charged with the same charge as the charged drug molecules (e.g., both are shown “positively” charged) in the formulation. The device is surrounded by a fibrous tissue capsule and microvasculature/capillaries, and a positively charged drug molecule is released from the reservoir (on the right side), and polarized electrodes drive the charged drug out, with the direction of the drug molecules being generally toward the oppositely charged electrode 58 located some distance away from the opened reservoir.

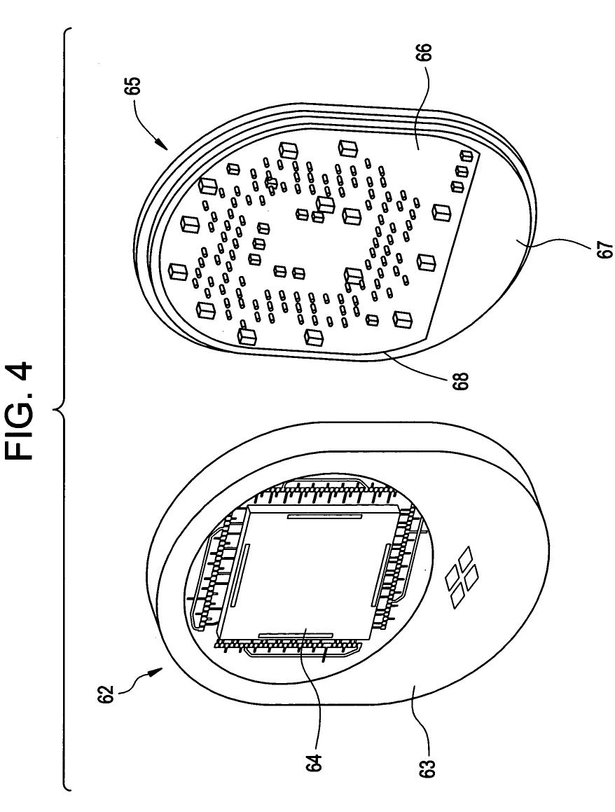

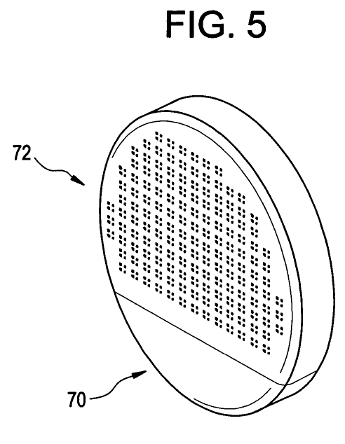

FIG. 4 and FIG. 5 illustrate two possible configurations of implantable drug storage and delivery devices. On the left, FIG. 4 shows the exterior of device 62, which includes a titanium hermetic enclosure 63, and the release side/surface of the body portion 64 that includes the reservoirs containing a drug formulation. On the right, FIG. 4 shows the interior portion 65 of the device, which includes microprocessor 66, battery 67, and wireless telemetry antenna 68. FIG. 5 shows another embodiment of the device which includes a first portion 72 that includes the reservoirs containing the drug formulation, and a second portion 70 that includes all of the control elements (e.g., electronics, power supply, wireless telemetry, etc.).

Representative examples of implantable devices that could be adapted for use with the drug formulations described herein include implantable pumps (e.g., mechanical pumps like those made by Medtronic-MiniMed and Arrow, or osmotic pumps like DUROS™ or Viadur™), and microchip chemical delivery devices and microchip biosensor devices (e.g., U.S. Pat. No. 5,797,898, U.S. Pat. No. 6,527,762, U.S. Pat. No. 6,491,666, U.S. Pat. No. 6,551,838, and U.S. Pat. No. 6,849,463).

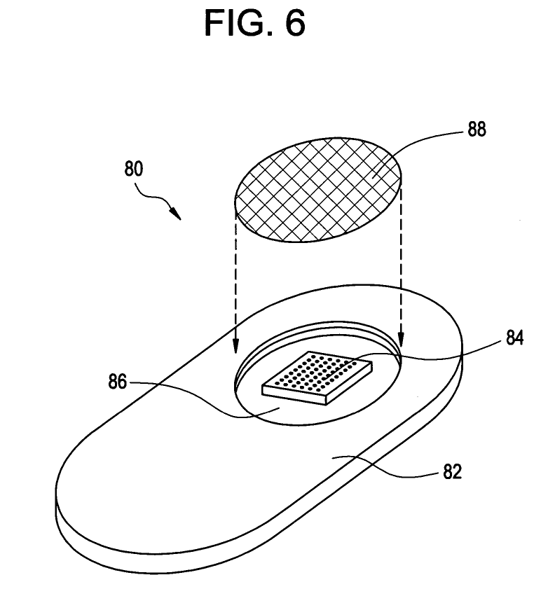

In one embodiment, the device includes a protective mesh located over at least the surface of the device in which the reservoirs are provided. The protective mesh can protect the reservoir caps from premature rupture, for example due to random mechanical forces directed against the face of the device before, during, or after implantation. The protective mesh should be substantially rigid to provide this protective function. For example, it can be formed of a biocompatible metal, polymer, or ceramic. Optionally, the protective mesh may be coated with one or more agents that promote vascularization, and/or minimize capsule thickness, at this reservoir portion of the device. In one embodiment, for example, the mesh is coated with angiogenic agents, anti-inflammatory agents, or both angiogenic agents and anti-inflammatory agents. One example is shown in FIG. 6, wherein the device 80 includes a titanium housing 82 (which contains inside the electronics, power source, and telemetry components), a drug delivery body portion 84 (which includes the reservoirs and drug or other molecules for storage and controlled release), a hermetic feed through 86, and a protective mesh 88. The protective mesh, although shown separated from the housing, will be attached in a secure manner, e.g., by welding, to the housing or to the hermetic feed through. The pores in the mesh could be tailored to induce tissue ingrowth as well.

Measurement of Cross-Tissue Capsule Transport

In another aspect, testing devices and methods have been developed as a means to better understand the impact of different formulation and device modifications on cross-tissue capsule transport. Importantly, the devices and methods can isolate and test the effect of tissue encapsulation—without the need to disrupt an intact tissue capsule. The devices advantageously allow access to the inside of intact tissue capsules in situ (in the animal), which, significantly, makes it possible to obtain detailed in situ measurements of molecular transport across tissue capsules. The devices allow one to assess methods for modifying/modulating the properties or structure of the tissue capsule (e.g. thickness, vascularity, density, porosity, permeability, etc.) and to make quantitative comparisons of different strategies for improving transport—for example comparing two tissue capsules that have been formed in different ways or under the influence of different conditions.

The devices also permit one to test different materials or device configurations. In one embodiment, the purpose of accessing the inside of the capsule is to test a device. For instance, one could compare devices that include means of opening a pathway into the device at a specific time, e.g., by mechanically rupturing, by electrochemically or electrothermally disintegrating, or by otherwise removing, a reservoir cap from an opening in the device body. In another embodiment, the purpose is to test a bulk material that is being considered for an implant material and observe what kind of capsule forms and whether such a material/device possibly would be useful as or in a drug delivery or biosensing device.

While these devices and methods are particularly applicable for assessing the impact on a reservoir-based (particularly microreservoir) implantable medical device, the devices and methods are not dependent, for example, on the reservoir contents, e.g., construction or membrane. The devices and methods can be used or adapted to measure transport in either direction: into the reservoir (e.g., for sensing applications) or from the reservoir (e.g., for drug delivery applications).

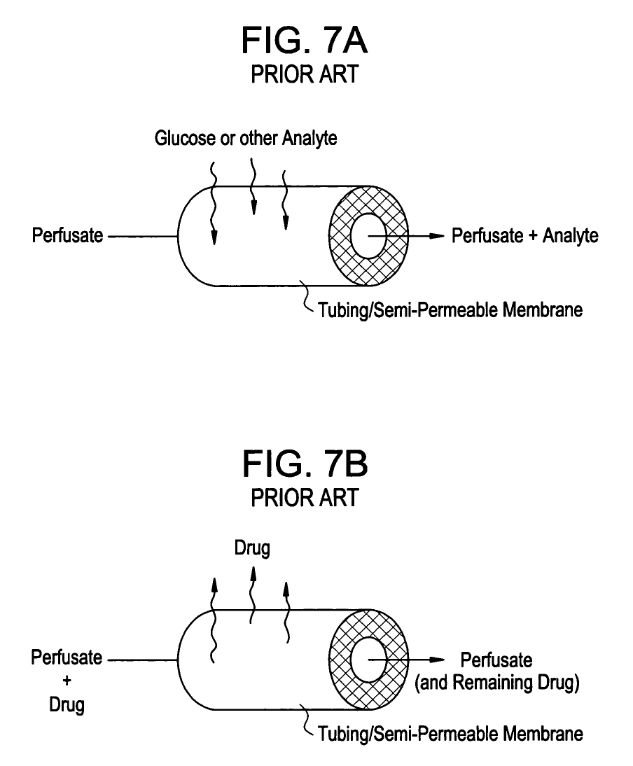

The test devices and methods are derived from the generic concept of microdialysis where a perfusate is flowed through tubing constructed of a semi-permeable membrane material, and a select molecular species flows to or from the perfusate through the tubing. See FIGS. 7A-B. The transported molecular species can be collected or measured, for example by measuring the amount of the molecular species (e.g., glucose or other analyte) that is in the perfusate after flowing through the tube, or by measuring how much of the molecular species (e.g., drug) that has left the perfusate after flowing through the tube. However, this conventional microdialysis process is unsuitable for extended periods of transport observations, because after a few days or weeks, a tissue capsule will form around the semi-permeable membrane tubing and will grow into the pores of the membrane. This encapsulation and ingrowth hinders transport through the membrane and can facilitate degradation of sensor enzymes contained in or just below the membrane, resembling the biofouling of sensor membranes that is one of the two primary reasons for failure of implantable sensors. (The other primary reason is time lag introduced by the capsule.)

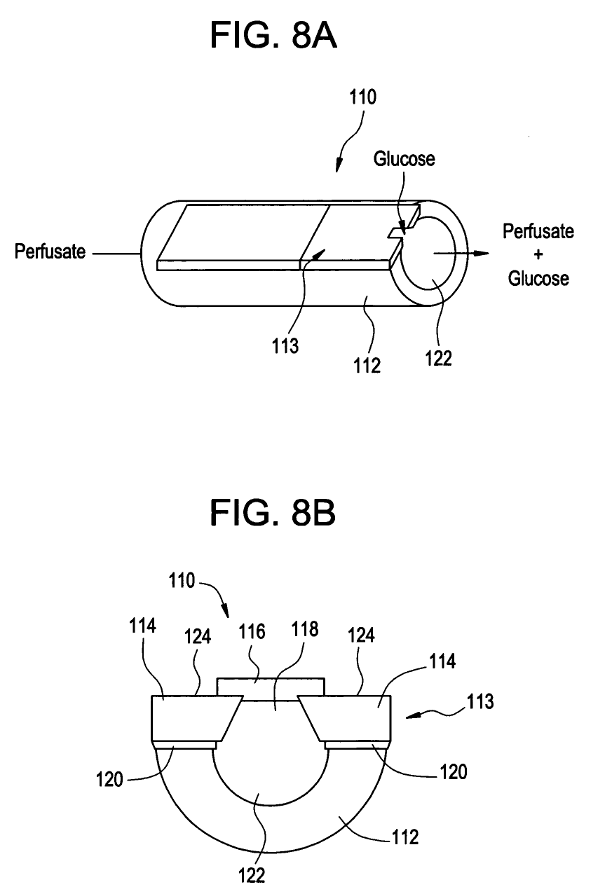

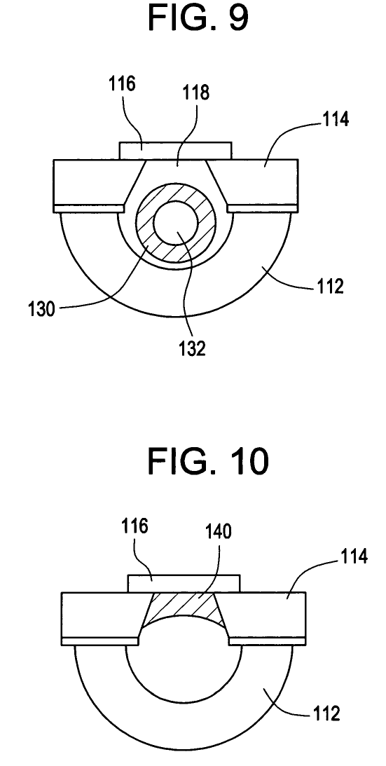

In one embodiment of the present testing devices and methods, the device includes a combination of a reservoir-based implantable medical device and an impermeable tubing, so that all transport to/from the perfusate is via the reservoir openings in the medical device, as illustrated in the non-limiting examples shown in FIGS. 8-14. In an alternative embodiment, the device includes a semipermeable material in combination with a reservoir-based implantable medical device, as illustrated in the non-limiting examples shown in FIGS. 9-10. For example, the semi-permeable membrane may be a typical microdialysis membrane that blocks convection while allowing diffusion of species with molecular weights less than about 20 kDa. The membrane could be inside or outside of the device, depending upon how long it is expected to function following implantation. Whether the apparatus needs a semi-permeable membrane depends for example on how well the tissue capsule is adhered to the device and/or how flexible or elastic the tissue capsule is, factors that impact fluid flow resistance from the tube to the into the capsule.

In some embodiments of the testing devices and methods, the openings in the tubes may be referred to herein as “reservoirs” even when they are not sealed/closed at both surfaces, e.g., where the interior opening is open to the tube lumen. That is, the term “reservoir” can be a closed volume in which another material is stored, or it can simply be the opening in the tube wall through which the inside of the capsule is accessed. Material transport from these reservoirs is designed to be essential diffusion-based with no or minimal convection.

FIGS. 8A-B illustrate one embodiment of a portion of the testing device. Device 110 includes tubing 112 which is attached to sensor package 113, which has outer surface 124. Perfusate flows in channel 122 which extends between the tubing 112 and the package 113. The sensor package includes substrate 114, reservoirs 118 (one is shown), and reservoir cap 116. The tubing 112 is secured to the substrate with impermeable, biocompatible material 120, which can be an adhesive or other sealing material known in the art. See, e.g., U.S. Pat. No. 6,730,072, U.S. Pat. No. 6,827,250, and PCT Publication No. WO 2005/010240. The tubing can be essentially any analyte- or metabolite-impermeable, biocompatible material. It preferably is relatively flexible and suitable for implantation into an animal.

In one embodiment, the device includes a semipermeable barrier structure blocking bulk fluid flow through the reservoir openings following reservoir cap disintegration or removal. FIGS. 9 and 10 include a semipermeable material interposed between the perfusate and the reservoir cap and thus, when the reservoir cap has been activated (i.e., disintegrated) the semipermeable material is interposed between the perfusate and the environment. In FIG. 9, the semipermeable material is in the form of a tube 130 inside channel 122, and perfusate flows through the interior space 132 of tube 130 and not through any other space within channel 122. In FIG. 10, the semipermeable material is in the form of a plug 140 disposed in the reservoir opening 118. In another embodiment, the semi-permeable membrane could be incorporated directly into the substrate during the substrate fabrication process. For example, the membrane could be a porous silicon membrane.

The outer surface of the package (particularly adjacent the reservoir openings) optionally can be coated, textured, or otherwise modified to affect the tissue capsule structural properties. Exemplary modification strategies include (1) approaches to reduce protein adsorption; (2) hydrogel modifications employing adhesion ligands, growth factors, and tissue response modifiers, such as cytokines, heparin, metabolic intermediates, neutralizing antibodies, NSAIDs, and TGFB; and (3) local drug delivery strategies. In one embodiment, one or more angiogenic agents known in the art are attached to or otherwise coated on the device to enhance the vascularity of the tissue capsule. In another embodiment, a portion of the reservoirs of the transport measuring device are loaded with angiogenic agent, and/or anti-inflammatory agent, and then sealed, while another portion of the reservoirs remain unfilled and open to provide transport information. The angiogenic agent and/or anti-inflammatory agent then would be released during the healing process. As used herein, the term “angiogenic” refers to a material or molecules that promote and maintain the development of blood vessels and microcirculation around the implanted device. In one example, the device releases or is coated with a vasoinductive or angiogenic agent such as a vascular growth factor. Suitable growth factors of this type include as vascular endothelial growth factor (VEGF), platelet growth factor, vascular permeability factor, fibroblast growth factor, and transforming growth factor beta. In another embodiment, the device includes an exterior membrane or coating layer that exhibits angiogenic properties. These layers can be made for example of tetrafluoroethylene, hydrophilic polyvinylidene fluoride, mixed cellulose esters, and/or other polymers. In another embodiment, the device is coated with one or more anti-inflammatory agents, such dexamethasone, which for example can decrease the overall thickness of the fibrous capsule. In a further embodiment, the device is coated with a combination of dexamethasone and VEGF which can reduce inflammation and increase vascularity around the implanted device.

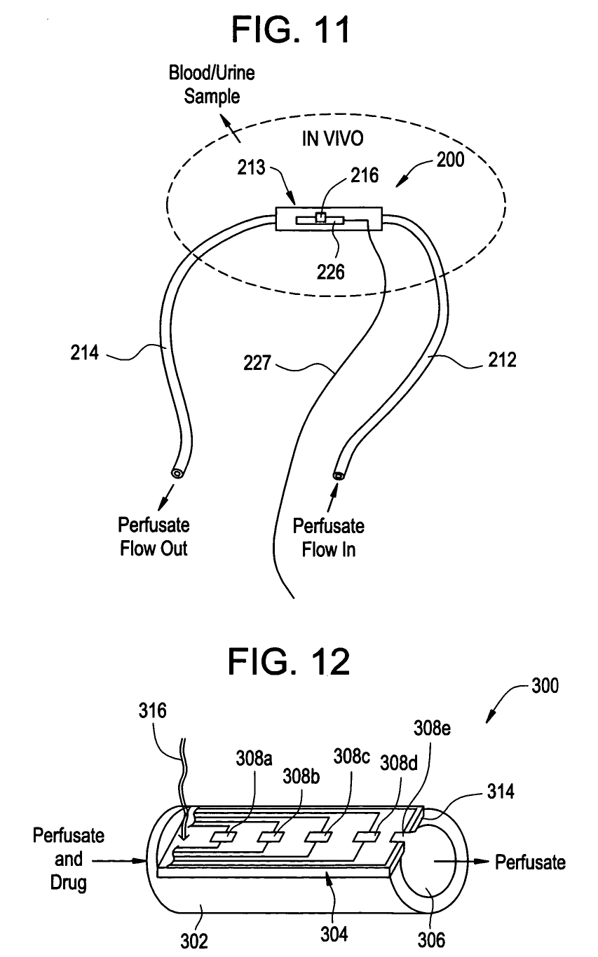

FIG. 11 is a plan view of a testing device 200 illustrating the perfusate flow therethrough. The dashed line represents the in vivo region, and shows which portions of the device would be implanted into an animal for testing and which parts would remain external in typical embodiments. Device 200 includes inlet tubing 212 and outlet tubing 214, tube/microchip component 213, with reservoir cap 216, electrical traces 226 and external wiring 227 for activating reservoir cap disintegration. In this embodiment, drug released from the device into the animal can be measure by periodic sampling of the animal's blood and/or urine. In a sensing study, one could give the animal a bolus of glucose, for example, and monitor how quickly the glucose permeates the capsule and reaches the sensor.

FIG. 12 shows part of a tube/microchip component 300 of a testing device. It includes tube body 302 and drug delivery package 304 attached thereto. Perfusate comprising a drug flows in channel 306. The drug delivery package includes substrate 314, reservoirs covered by reservoir caps 308a-e, electrical traces to/from the reservoir caps, and external wiring 316. A linear array of five reservoirs is shown. In a testing operation, a reservoir cap is disintegrated, thereby creating a flow path out of the device. Drug diffuses from the perfusate and toward/through adjacent tissue capsule in the body of the test animal.

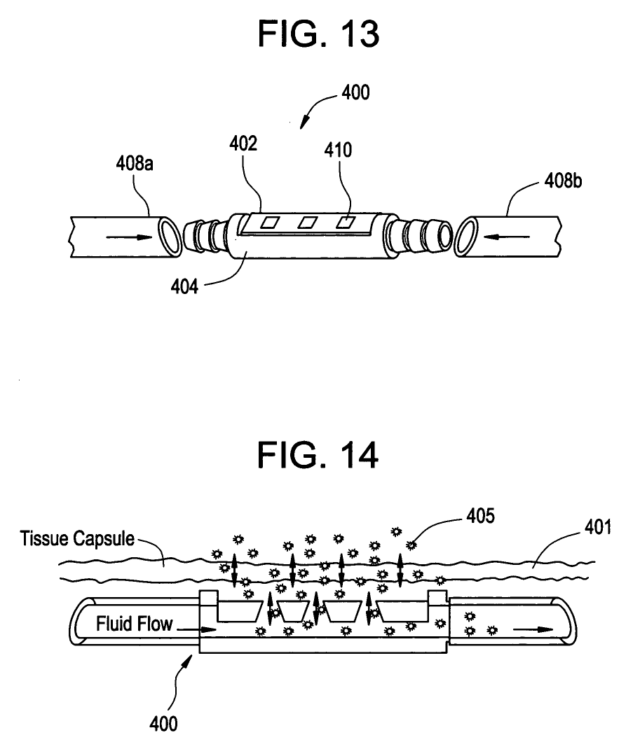

FIGS. 13 and 14 show another embodiment of a testing device 400 which includes microchip portion 402, rigid tube body 404, and flexible inlet/outlet tubing 408a and 408b. Tube body can be made of a biocompatible metal (e.g., stainless steel, titanium, etc.) or polymer. Reservoir membranes 410 are disposed in the reservoir openings. In operation, analyte 405 flows from the tissue capsule 401, through the reservoir openings, and into the perfusate, where the analyte can be measured.

In a typical test, one would allow the capsule to form for a specific period, and then open the reservoirs, and then make measurements for some period of time (e.g., from a few days up to two weeks), maybe longer depending on how rapidly capsule tissue begins to grow into and clog the opening.

While the devices and methods are primarily intended for use in non-human mammals, the devices and methods described herein could be used to test capsule permeability in humans.

The perfusate can be essentially any one suitable for a particular test application. For instance, it could be designed to simulate the drug contents of reservoir, or it could be a simple aqueous fluid receptive to diffusion of glucose. Representative examples of perfusate include PBS, another physiologic fluid or buffer, and a drug solution in a non-aqueous, biocompatible solvent. The perfusate can be pumped through the tubing of the device using essentially any pumping means known in the art including syringes, metering pumps, and the like.

In practice, the tubes of the testing device can, in one embodiment, be placed completely under the skin of the test animal at the time of implantation, and the skin at that site allowed to heal, e.g., to reduce the chance of infection at the site where the tubes are externalized. Then, when it is time to access the tubes and run an experiment, a small incision is made to access the tubes, e.g., the tubes could be partially extracted. For the duration (e.g., few days, or week) of the experiment, it is necessary to keep the site clean to avoid infection as with any other wound healing. In an alternative embodiment, the tubes remain externalized throughout the implantation period.

The present testing devices and methods are useful for studying the effect in vivo of tissue capsule formation on an implanted medical device. Advantageously, the devices and methods will isolate the effect of the encapsulation and would be independent of the particular reservoir contents of the implanted device. The devices and methods would for example enable one to quantify the glucose (or other analyte or drug) passing through both the tissue capsule that forms around the implanted medical device. The testing devices and methods can be designed to minimize any effect the device would have on molecular transport so that the results are indicative of the capsule.

The present devices and methods can be further understood by reference to the following non-limiting examples.

### EXAMPLE 1

### Testing Device Design

The testing device would be in the form of a closed loop implant test system. In this design, a microchip device will be attached along a length of metabolite impermeable tubing, substantially as shown in FIG. 13. The microchip will contain active reservoir caps that can be selectively disintegrated at any time following implantation. The design may include the placement of suture loops if necessary or the placement of surgical mesh to reduce implant motion which will disrupt the normal wound healing response. The microchip will be sealed to the test loop system, and will include the electrical system, used to activate the membranes and open the reservoirs and the percutaneous connectors.

The testing device will be implanted into the subcutaneous space of the animal model, and the incision allowed to heal for a pre-determined period of time. The wiring and tubing will be accessible through a percutaneous connector. Then, at selected times, the reservoir caps will be disintegrated by electrothermal ablation, in concert with a subcutaneous injection of a metabolite. The test loop system (i.e., the interior fluid) will be exposed to in vivo environment via the exposed reservoir opening. The metabolite will subsequently be transported between the in vivo environment and the test loop system, substantially as shown in FIG. 14. A saline solution will be pumped through the test loop system, removing the metabolite from the device under the reservoirs. The outlet saline solution will then be tested for the metabolite concentration.

### EXAMPLE 2

### Leak Testing

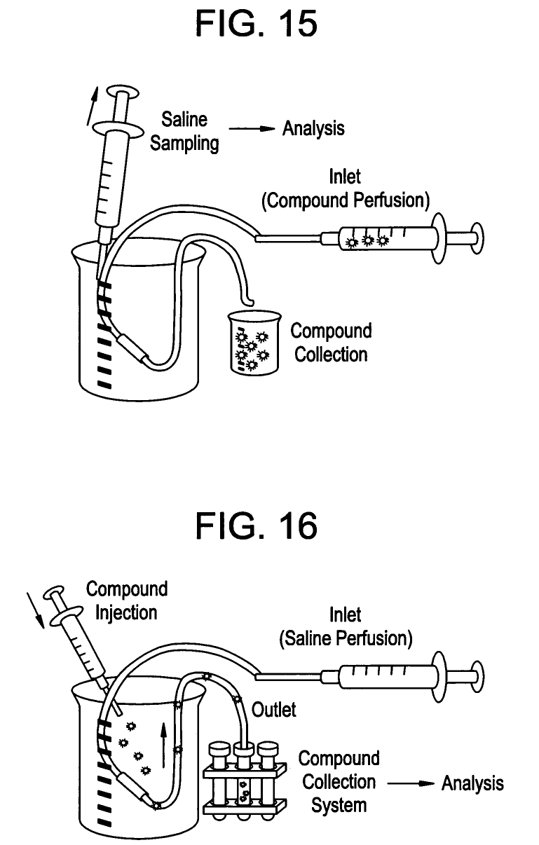

In vitro testing of the testing device to be used in animals is important prior to implantation. A system leak test will be performed. The device will be placed in a saline solution. The membranes of the device will remain intact throughout the experiment. An easily detectable compound (e.g. radio-labeled mannitol) will be pushed through the system using a pump. At pre-determined time-points, the saline will be sampled and analyzed for any evidence of the molecule pushed through the system. This experiment must be repeated on multiple devices to ensure proper device assembly. FIG. 15 illustrates the test set up.

### EXAMPLE 3

### In Vitro Testing

Prior to any in vivo studies, the device will be tested in vitro to ensure device functionality. The device will be placed in a saline solution. A saline solution will be pumped through the system. The microchip reservoirs will then be ablated, opening access to the test loop system. A specified amount of an easily detected compound will be injected into the saline solution in which the device is immersed. Saline solution will be pumped through the system and collected at certain time intervals. The outlet saline will be analyzed for the compound concentration at predetermined time-points. This experiment should be repeated on multiple devices to ensure proper device function. The in vitro test will provide a best case experiment for comparison purposes. FIG. 16 illustrates the test set up. Repeatable results should be obtained prior to in vivo experimentation.

In addition to testing functionality, these test methods will be useful for assessing the performance of a device or a particular transport enhancement feature. For instance, when the device is immersed in a solution having a concentration of Y, then one can determine the concentration that is recovered in the solute as a function of the flow rate, or for a given flowrate, which is driven by a hydrostatic pressure difference between the inlet and the outle, how much perfusate is lost through the reservoir opening.

### EXAMPLE 4

### In Vivo Testing

The test system will be implanted into the subcutaneous space. Post-implantation, the system will continuously be filled with a saline solution and periodically flushed out. When desired, the reservoir caps covering the microchip reservoirs will be electrically ablated. Prior to this on the testing day, a subcutaneous glucose sensor will be implanted subcutaneously to serve as a fresh control. Optionally, another multi-reservoir transport measuring device can be implanted the day of or the day before to get day “0” and day “x”readings. Immediately after ablation, an injection of the desired metabolite will be given subcutaneously (SC), intravenously (IV), intramuscularly (IM), and/or intraperitonealy (IP). At predetermined time-points after injection, the fluid in the system (˜1-2 ml) will be completely replaced with fresh solution. At these time-points, blood will be drawn and the metabolite level analyzed. This is to serve as a second control. After the last system sample is taken, the animal will be euthanized and the device and tissue capsule will be removed. A histological assessment of the capsule along with capsule vascularity quantification will be performed. This information will be compared to the transport quantities obtained after the metabolite injection.

The study design will include groups of multiple animals each appointed to a specific testing time-point. Each individual study will run for six months since capsule properties do not change after 3 to 6 months of implantation, if the implant properties and animal health remain the same throughout the implant period.

Initial studies will be conducted using a simple system of tubing, stainless steel and a silicon microchip. The data obtained provides a baseline without capsule modifiers. Once the baseline in vivo study is completed, multiple studies using various fibrous capsule modifiers will be conducted. For example, the impact of porous materials and the impact of local delivery of VEGF (injection or hydrogel) on capsule formation will be investigated. At all time-points, the injection test and histology data will be compared to the baseline implant study data.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

1. A method of enhancing the transport of drug from an implanted drug delivery device across a tissue capsule, the method comprising:
controllably releasing a drug formulation from a plurality of discrete reservoirs located in medical device implanted in a patient; and
controllably releasing an effective amount of a transport enhancer from said medical device implanted in a patient, to facilitate transport of the released drug formulation through a fibrous tissue capsule, if any, which exists around the device at the site of implantation.

2. The method of claim 1, wherein the release of the enhancing agent is from one or more reservoirs located in the device.

3. The method of claim 1, wherein the release of the enhancing agent is from a surface coating on the device.

4. The method of claim 1, wherein the release of the transport enhancer occurs concurrently with release of the drug formulation.

5. The method of claim 1, wherein the release of the transport enhancer occurs continuously.

6. The method of claim 1, wherein the drug formulation further comprises the transport enhancer, and the drug formulation and the transport enhancer are released from the same reservoirs.

7. The method of claim 1, wherein the transport enhancer comprises a solvent or co-solvent for the drug.

8. The method of claim 1, wherein the transport enhancer comprises a surfactant.

9. The method of claim 1, wherein the transport enhancer comprises dimethylsulfoxide or N-methylpyrrolidone.

10. The method of claim 1, wherein the drug molecules comprises charged molecules and the transport enhancer comprises ion-pairing counter-ions.

11. The method of claim 1, wherein the transport enhancer comprises molecules which dissolve or degrade components of the tissue capsule.

12. The method of claim 11, wherein the molecules comprise collagenase.

13. The method of claim 11, wherein the molecules comprise thrombin, fibrinolysin, hyaluronidase, or trypsin.

14. The method of claim 1, wherein device further includes means for mechanically driving the drug formulation out of the reservoir and through the tissue capsule.

15. The method of claim 14, wherein the means for mechanically driving the drug formulation comprises a piston, a water-swellable material, or a combination thereof.

16. The method of claim 1, wherein the device further comprises an angiogenic coating or angiogenic molecules for release.

17. The method of claim 16, wherein the angiogenic coating, angiogenic molecules for release, or both, comprise a vascular endothelial growth factor.

18. The method of claim 1, wherein the device further comprises an anti-inflammatory agent, which is released from the reservoirs or from a coating on the device or both from the reservoirs and the coating.

19. The method of claim 18, wherein the anti-inflammatory agent comprises dexamethasone.

20. A method of enhancing the transport of drug from an implanted drug delivery device and across a tissue capsule, the method comprising:
controllably releasing a drug formulation, which comprises charged drug molecules, from a plurality of discrete reservoirs of a medical device implanted into a patient, the release of the drug and the release of the enhancing agent being from one or more reservoirs located in the device; and
utilizing an electromotive method to enhance transport of the charged drug molecules through a tissue capsule, if any, surrounding the implanted medical device.

21. The method of claim 20, wherein the electromotive method comprises iontophoresis.

22. The method of claim 20, wherein an external surface of the medical device is charged by an electronic component therein, or thereon, creating a driving force effective to propel the drug molecules through a tissue capsule, if any, surrounding the implanted medical device.

23. A method of enhancing the transport of an analyte to a sensor device implanted in a patient, the method comprising:
controllably releasing an effective amount of a transport enhancer from the implanted sensor device, said device comprising a plurality of discrete reservoirs having sensors located therein.

24. The method of claim 23, wherein the device further comprises:
reservoir caps; and
means for rupturing said reservoir caps.

25. An implantable medical device comprising:
a body portion;
two or more reservoirs located in and defined by the body portion;
reservoir contents in the reservoirs; and
means for enhancing mass transport, of all or a portion of the reservoir contents or of an environmental component intended for contact with all or a portion of the reservoir contents, through any fibrous tissue capsule that may form around the device following implantation.

26. The device of claim 25, wherein the reservoir contents comprises a drug formulation.

27. The device of claim 25, wherein the reservoir contents comprises a sensor.

28. The device of claim 25, wherein the means for enhancing mass transport comprises a transport enhancer, an electromotive device, a positive displacement mechanism, or a combination thereof.

29. The device of claim 25, further comprising an angiogenic coating or angiogenic molecules for release.

30. The device of claim 29, wherein the angiogenic coating, angiogenic molecules for release, or both, comprise a vascular endothelial growth factor.

31. The device of claim 25, further comprising an anti-inflammatory agent, which is released from the reservoirs or from a coating on the device or both from the reservoirs and the coating.

32. An implantable device for testing drug or analyte transport through a tissue capsule, the device comprising:
a primary body having an outer surface, a perfusate fluid inlet, a perfusate fluid outlet, and a fluid conduit extending between the inlet and the outlet;
a substrate attached to the primary body;
at least one reservoir defined in and extending through the substrate, the reservoir having a first opening in the fluid conduit and a second opening which can be open to the outer surface of the device;
at least one reservoir cap covering the second opening of the reservoir;
means for selectively disintegrating or removing the reservoir cap.

33. The device of claim 32, further comprising a first flexible tubing connected to the perfusate fluid inlet, a second flexible tubing connected to the perfusate fluid outlet, and a means for flowing perfusate thorough the fluid conduit and the flexible tubings.

34. The device of claim 34, further comprising a semipermeable barrier structure blocking bulk fluid flow through one or both of the reservoir openings following reservoir cap disintegration or removal.
